(19) United States
(12) Patent Application Publication
(10) Pub. No.: US 2014/0144437 A1
(43) Pub. Date: May 29, 2014

(54) Medicament transport container and method of use

(71) Applicant: Nancy T. Miller, Chicago, IL (US)

(72) Inventor: Nancy T. Miller, Chicago, IL (US)

(21) Appl. No.: 13/694,371

(22) Filed: Nov. 26, 2012

(51) Int. Cl. A61B 19/00; A61M 5/31; A61M 15/00; A45C 11/00

(52) U.S. Cl. CPC A61B 19/44; A45C 11/00; A61M 5/31; A61M 15/00
USPC 128/203.12; 206/570; 206/571; 604/506

(57) ABSTRACT

A multiple medicament container and method for retaining a plurality of medicaments for students on excursions from school is disclosed. The container is configured to securely hold medicaments of a variety of configurations including epinephrine pens and asthma inhalers as well as other forms. The container also holds cards identifying medical conditions relating to each student for which medicaments are provided as well as cards denoting administration protocols for each medicament loaded into the container. A single medicament embodiment of the container is also disclosed wherein the container is already paired to the subject student.

### FIELD OF THE INVENTION

The instant invention relates to containers for transporting pluralities of differing medicaments as well as associated medical sundries and a method of use thereof.

### BACKGROUND OF THE INVENTION

Children having medical conditions are frequent users of medicaments in a school environment. Often, in a school setting these medicaments are stored at the station of the school nurse. Such medicaments take a variety of forms and are for both chronic conditions such as insulin for diabetes as well as acute crises such as injectable epinephrine for allergic reactions. In a school environment these medicaments are well controlled, both in their distribution as well as in having their proper storage assured. In a school nurse's station such medicaments are located in a secure location and care is taken that each medication is carefully labeled and stored to assure that the right medication is delivered to the correct student. Additionally each medicament is stored so that degradation of the medicament is prevented. Such prevention takes the forms of refrigerating medications like insulin that need to be cooled, keeping light-sensitive medications in a darkened storage area, maintaining temperature control for those medications that may be stored at room temperature but within a specified limit or the like.

All of these carefully crafted controls are much more difficult or nearly impossible to achieve once students having medical conditions leave the school premises for field trips; outings or other supervised extracurricular activities off the school grounds. Even though allergic students run a higher risk of being exposed to allergens, diabetic students run a higher risk of their glucose exceeding tolerable limits and other students each having any of a multitude of medical conditions having their respective conditions exacerbated by being in an uncontrolled environment, there presently exists only the most rudimentary methods for storing and transporting student medicaments to these various events. Bulky coolers are itinerantly used to transport medicaments that require cooling and a variety of non-specific carryalls are used to transport medicaments and other sundries in an inefficient and disorganized way.

### SUMMARY OF THE INVENTION

The instant invention provides for an organized, safe and effective way to transport medicaments by responsible school officials including instructions for the use of such medicaments should the need arise in an environment outside of the school. Additionally the invention provides for a method of use for such a transport apparatus to assure maximizing efficacy in the administration of such medicaments to the right patient, in the right dose, via the right means of administration at the right time. Also the invention provides for a way to enter and record notes relating to such administration as well as providing an organized way to maintain instructions for the administration of such medicaments. The invention further provides for a transport apparatus for medicaments so configured to assure that medicaments are transported at an allowable temperature for extended periods without refrigeration. These and other aspects of the invention will become apparent in the Detailed Description of the Preferred Embodiment and Claims appended hereto.

### BRIEF DESCRIPTION OF THE DRAWINGS

FIG. 1 is a perspective view of the container.

FIG. 2 is a plan view of the interior of the container showing the inside cover.

FIG. 3 is a plan view of the medial divider of the medicament container with cutaway showing the interior thereof.

FIG. 4 is a plan view of the interior rear compartment.

FIG. 5 is a cutaway view of the front cover.

FIG. 6 is a diagram of an Emergency Information Card.

FIG. 7 is a diagram of an Action Plan card.

FIG. 8 is a diagram of a Medication Sheet.

FIG. 9 is a perspective view of the single medicament embodiment if the invention.

### DETAILED DESCRIPTION OF THE PREFERRED EMBODIMENT

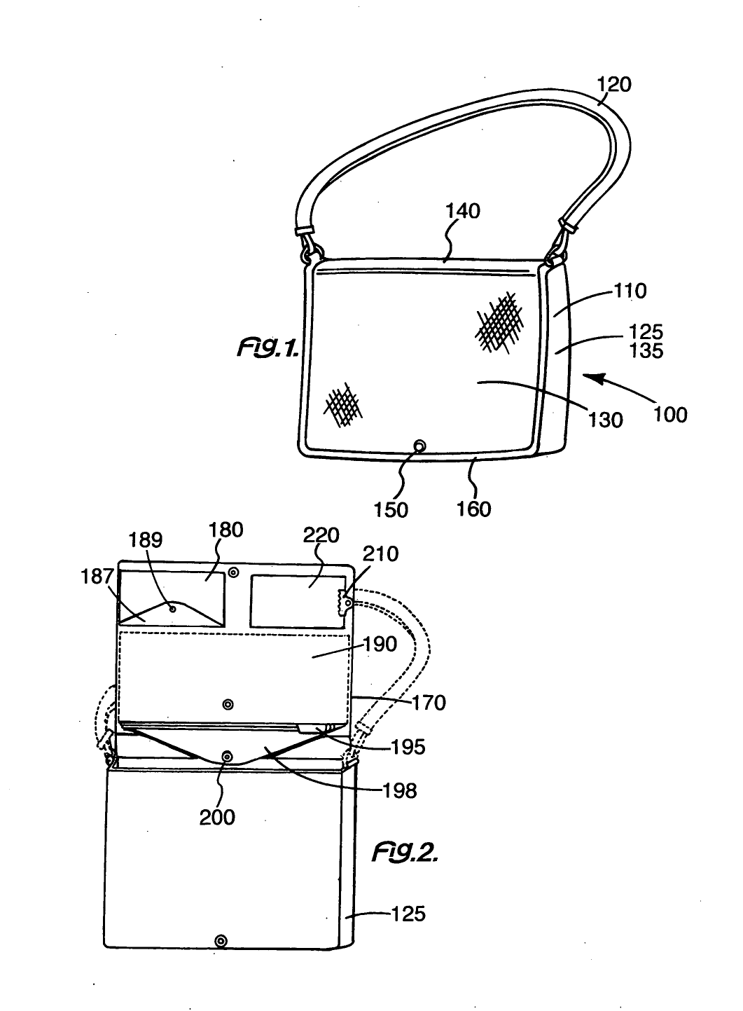

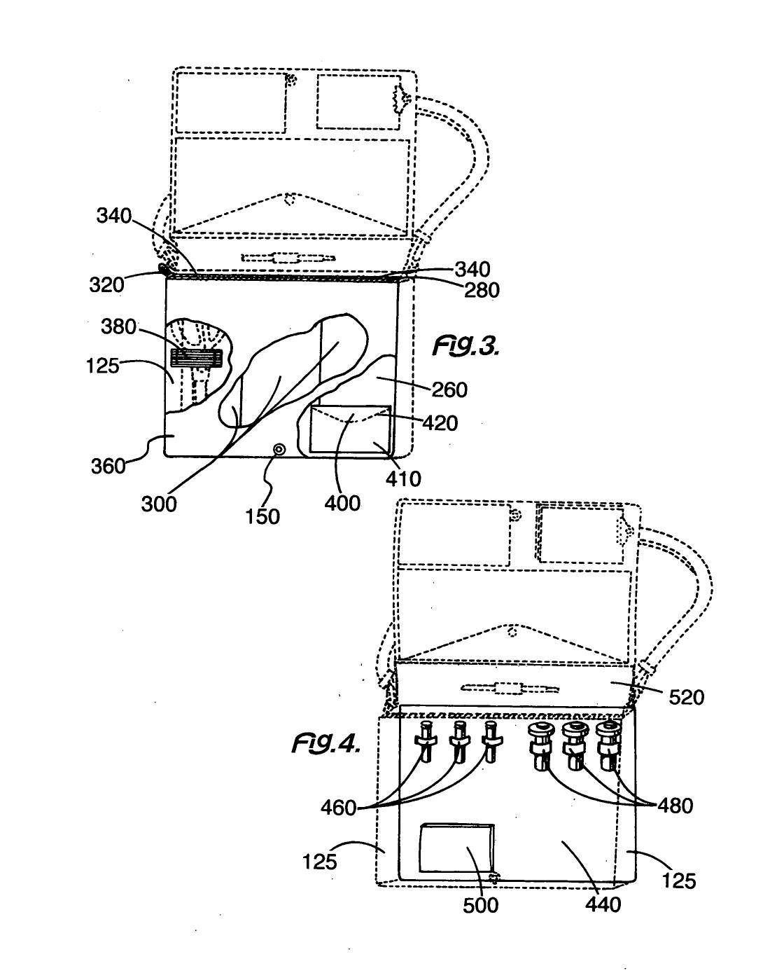

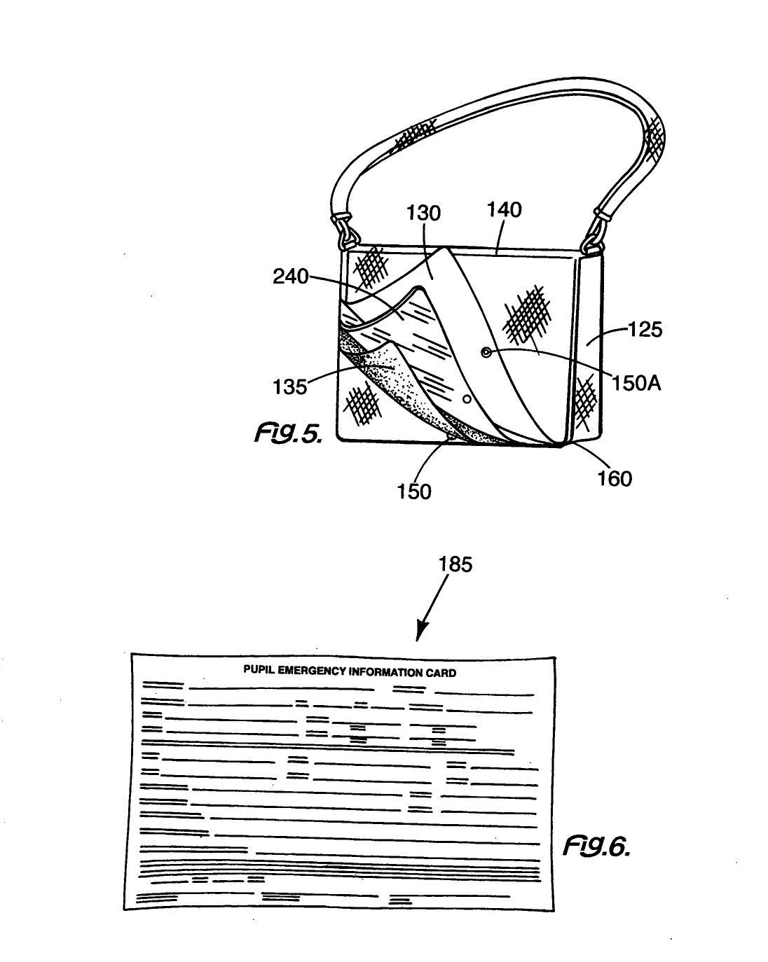

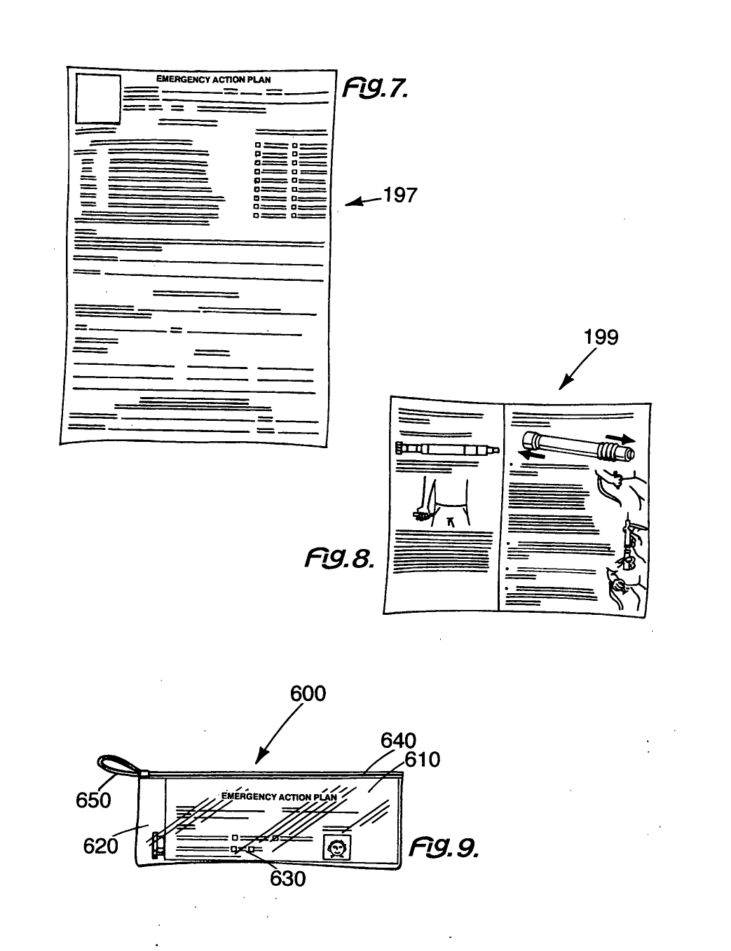

As shown if FIG. 1 the container 100 comprehends a body 110 and a handle or strap 120. The body, 110 having a cover 130 attached thereto by the cover's 130 upper edge 140 which may be made integral with the body 110. Cover 130 and body 110 are preferably made of a sturdy synthetic material which is water resistant and contains insulating material 135. Cover 130 additionally has a closure 150 attached near the distal edge 160 of the cover 130. This closure is operative to releasably affix the cover 130 to the body 110 on a closed position. Preferably, closure 150 is magnetic and therefore corresponds to a ferromagnetic insert 150A shown in FIG. 5. As shown in FIG. 2, Cover 130 has an interior side 170 which comprehends a first pocket 180 which is configured to accept standard student emergency information cards 185. These emergency information cards 185, as best shown in FIG. 6 contain student specific information directing the school official carrying the container 100 which student should be receiving which medicament contained in the container 100 as well as proper dosing and administration instructions for the particular student for which the medicament is to be used. First pocket 180 also comprehends a flap 187 and a closure 189 to insure that the contents 185 are not accidently lost. The cover 130 also features a second pocket 190 which includes interior dividing tabs 195 which operate to separate materials contained therein. These materials preferably consist of emergency action plans 197 as shown in FIG. 7 which instruct the school official using the container in the correct procedures for caring for students having conditions needing specialized attention. The materials carried in second pocket also preferably comprehend medication sheets 199, as shown in FIG. 8, which describe the medicaments contained in the container 100. The second pocket 190 also features a flap 198 and a closure 200 which operate similarly to the flap 187 and closure 189 of the first pocket 180. Cover 130 also features a retainer 210 affixed along the perimeter of the cover 130 opposite the first pocket 180 which is operative to retain paper or note cards 220 to allow the school official to keep notes on emergency situations which occur while the container 100 is in use or to take notes of medications administered to inform the school nurse or student's parent of medicaments administered by the school official.

As best shown in FIG. 5, the front cover 130 further comprehends an interior platen 240 which completely subtends the area of cover 130 and is adhered to the outer skin 105 of the cover 130. In operation, the platen 240 provides for a planar surface on the exterior of cover 130 having sufficient smoothness and stiffness, to provide for a writing surface thereon.

Interior to cover 130 resides a medial divider 260. The medial divider 260 serves to divide the container 100 into two separate sections transversely along the long axis thereof. The medial divider 260 further features an interior volume 280 which is preferentially subdivided into three vertical sections 300. These vertical sections 300 are accessed by a closure 320 which resides along the free edge 340 of the medial divider 260. The other three edges being firmly affixed to the side walls 125 of the container 100. These vertical sections 300 provide for storage of miscellaneous items necessary or desirable to be carried with the various other medicaments or medical sundries contained within the container 100. The distal wall 360 of the medial divider 260 further features an elastic retainer 380 adapted to receive and retain a pair of scissors and diagonally opposite thereto the distal wall 360 further has defined thereon an insulated pocket 400 adapted to receive at least one container of temperature sensitive medicament. Insulated pocket 400 additionally has a flap closure 410 to seal the interior thereof. The flap closure 410 is preferentially sealed to the insulated pocket 400 via a hook and loop closure 420. Additionally, all sides of the insulated pocket 400 and including the flap closure 410 are filled with insulating material between the exterior skin and interior skin defining the pocket 400 and flap closure 410.

Moving toward the rear interior wall 440 of the container 100, the rear interior wall 440 comprehends a first set of loop retainers 460 adapted to receive epinephrine pens. The first loop retainers 460 are disposed near the top edge of the rear interior wall 440 so as to be easily accessible to school personnel as a timely administration of epinephrine is critical in an emergency. Laterally adjacent to first loop retainers 460 are second loop retainers 480 which are adapted to receive aerosol inhalers for treating asthma and the like. As with the epinephrine pens since quick access to these aerosol inhalers is of primary importance so the second loop retainers 480 are also disposed near the top edge of the rear interior wall 440. Below the first loop retainers 460 is a rear wall pocket 500 which is adapted to receive and retain adhesive bandages of various sizes. Top web 520 which connects the exterior cover 130 and rear interior wall 440 additionally features a third loop retainer adapted to receive and retain a pen.

An alternative embodiment of the instant invention as shown in FIG. 9 is adapted to retain a single emergency medicament. Container 600 comprehends an interior pocket 610 and a sidewall 620 which has a window 630 defined thereon. Sidewall 620 is preferably of a bright color so as to alert school officials of the presence of the container. This coloration is preferred since in the case of the single medicament container, the normal mode of transport of the container is by the patient rather than the school official. Interior to the window 630 is a document pocket 640 adapted to retain an emergency information card 185 and an emergency action plan 197 for the single medicament and individual to which the medicament pertains. As a single medicament is most likely to be used in a crisis situation such as anaphylaxis, immediate access to the information on the card 185 and action plan 197 is specifically desired. The top edge of the container 600 has a closure defined thereon to selectively close the interior pocket 610 to retain the medicament therein. Adjacent to the closure is an affixment 650 to attach the container 600 to a backpack or the like. Operatively this alternative embodiment is used similarly to the multiple medicament container as subsequently described with the differences being that the single medicament embodiment of the container 600 is already paired to the student or patient to which the medicament within the container pertains therefore allowing the school official or employee to administer the medicament immediately upon identifying the student with the information contained in the individuated container. Additionally, in a transport situation such as a bus or the like, the single use container could be paired to an identifying card carried on the bus and accessible to the driver identifying the student as having an allergy and carrying a single medicament container.

In use the instant container 100 is loaded with appropriate medicaments for the students for which the instant container 100 is scheduled to accompany outside of the school environment; for example on a field trip. Additionally, corresponding to the students and their medications, a plurality of emergency information cards 185 corresponding to each of the students related to the instant container 100 as well as a corresponding plurality of emergency action plans 197 and medication sheets 199, each of which corresponds to an individual student related to the medicaments in the container 100.

Subsequent to loading the container 100, the container 100 accompanies the students on their activity outside of the school environment. Should the need arise and a student fall ill, the appropriate school staff member having possession of the container 100 opens cover 130 and looks in first pocket 180 and retrieves the appropriate emergency information card 185 for the specific student and identifies the appropriate medicament contained in the container 100. Then, the school official retrieves the appropriate emergency action plan 197 from second pocket 190 which, as said, contains the administration instructions for delivering the particular medicament. The school official then administers the medicament to the student and notates such administration on the action plan 197 and then contacts the appropriate emergency personnel and school officials per the procedure on the action plan 197.

This detailed description is merely indicative of the presently preferred embodiment of the instant invention and is not to be construed as limiting the scope thereof in any way beyond that recited in the Claims appended hereto.

Therefore, in accordance with my invention, I claim:

1. A transport container for a plurality of medicaments comprising an outer body comprehending a cover affixed to the body along one edge of said cover and extensive along a front face of said body, said cover having a rigid interior platen subtending essentially the full extent of said cover and said cover having an interior facing side thereto wherein said interior facing side comprehends at least one pocket adapted to receive at least one card containing indicia relating to at least one patient for which medicaments contained within the container are indicated; said body defining an interior thereof wherein said interior is further subtended by a transverse medial web extensive along a long axis of said interior, said transverse medial web comprehending a plurality of flexible pockets adapted to receive said medicaments.

2. The container of claim 1 and said cover having an exterior surface of fabric wherein said fabric and said interior platen are co-adapted to provide a smooth surface suitable for writing.

3. The container of claim 2 and said cover further comprising a magnetic closure adapted to releasably affix said cover in a closed position against body.

4. The container of claim 3 and said cover comprehending a plurality of pockets adapted to receive, in a first pocket, information cards; said information cards denoting a specific medical condition pertaining to an individual for which medicaments are contained in said container and a second pocket adapted to receive action plan cards wherein said action plan cards contain information pertaining to administration and dosing of such medicaments contained in said container.

5. The container of claim 1 and said interior having a rear wall wherein said read wall is distal to said cover and said rear wall comprehends a plurality of flexible loops adapted to receive cylindrically containerized medicaments.

6. The container of claim 5 and said cylindrically containerized medicaments are asthma inhalers.

7. The container of claim 5 and said cylindrically containerized medicaments are epinephrine pens.

8. A multiple medicament container for transporting medicaments delimited to specified individuals comprising: body means for enclosing an interior space adapted to receive medicaments; retaining means disposed within said interior space for holding containers of medicaments; first information means for retaining indicia relating to said specified individuals denoting medical conditions which said medicaments are adapted to treat, releasably retained within said body means; second information means for retaining indicia relating to specific medicaments retained by said retaining means releasably retained within said body means, and cover means, affixed to said body means, for isolating said interior of said body means from an external environment.

9. The container of claim 8 and said cover means for isolating said interior of said body means further comprising rigidifying means for stiffening said cover means so that said cover means can support a writing surface placed thereon.

10. The container of claim 9 and said body means further comprising a plurality of pocket means for receiving said first information means and said second information means.

11. The container of claim 10 and said body means further comprising interior web means, disposed within said interior, for subdividing said interior into a plurality of areas.

12. The container of claim 11 and said web means further comprising web pocket means for retaining items within said web.

13. The container of claim 12 and said web pocket means further comprising closure means for selectively isolating said web pocket means from said interior.

14. The container of claim 13 and said cover means further comprising closure means for releasably affixing said cover means to said body means in a specified position.

15. A method of using a medicament container for dispensing at least one medicament to a specified individual comprising:
providing a container adapted to hold the at least one specified medicament relating to said specified individual;
providing a first card containing indicia relating to medical conditions appurtenant to at least said specified individual, said first card disposed within said container;
providing a second card containing indicia relating to administration protocols said at least one medicament retained in said container, said second card disposed within said container;
identifying the medical condition retained on said first card relating to the specified individual for which assistance is necessary;
retrieving the indicated medicament from said container identified on said first card relating to said specified individual;
retrieving said second card relating to an administration protocol for the indicated medicament;
preparing to administer said indicated medicament according to said protocol; and
administering the medicament.

16. The method of claim 15 further comprising said first card being one of a set of said first cards and said second card being one of a set of said second cards.

17. The method of claim 16 and said medicament container containing a plurality of medicaments relating to a plurality of individuals as identified in said set of first cards.

18. The method of claim 17 and said set of second cards corresponding to a plurality of administration protocols for said plurality of medicaments.

19. The method of claim 16 and said container further comprising providing a writing surface adapted to support said notation.

20. The method of claim 17 and providing indicia on each of said medicaments correlating said medicaments to each of said specified individuals for which the medicament is indicated.
